(19) United States
(12) Patent Application Publication
(10) Pub. No.: US 2017/0312347 A1
(43) Pub. Date: Nov. 2, 2017

(54) Methods For Treating Cancers Using Ace Inhibitors, ARB, or Celecoxib and Olmesartan

(71) Applicant: Autotelic LLC, City of Industry, CA (US)

(72) Inventor: Vuong Trieu, Agoura Hills, CA (US)

(73) Assignee: Autotelic LLC, City of Industry, CA (US)

(21) Appl. No.: 15/581,868

(22) Filed: Apr. 28, 2017

Related U.S. Application Data
(60) Provisional application No. 62/329,985, filed on Apr. 29, 2016.
(60) Provisional application No. 62/450,545, filed on Jan. 25, 2017.
(60) Provisional application No. 62/480,273, filed on Mar. 31, 2017.

(51) Int. Cl. A61K 38/55; A61K 31/4184; G01N 33/574; A61K 38/54; A61K 31/00

(52) U.S. Cl. CPC A61K 38/556; G01N 33/574; A61K 38/54; A61K 31/4184; A61K 31/00

(57) ABSTRACT

Methods for treating cancers, such as breast cancer or skin cancer, using an ACE or ARB, or a combination of celecoxib and olmesartan.

### CROSS REFERENCE TO RELATED APPLICATIONS

The present application claims priority to U.S. Ser. No. 62/329,985, filed Apr. 29, 2016, U.S. Ser. No. 62/450,545, filed Jan. 25, 2017, and U.S. Ser. No. 62/480,273 filed Mar. 31, 2017, herein each incorporated by reference in their entirety.

### FIELD OF THE INVENTION

The present invention relates to compositions and methods for treatment of proliferative disease. In specific aspects, the present invention relates to compositions including celecoxib and olmesartan in combination; methods for treatment of a proliferative disease including administration of celecoxib and olmesartan to a subject in need thereof; and particularly methods for treatment of cancer including administration of celecoxib and olmesartan to a subject in need thereof. In other aspects the invention comprises using an ACE inhibitor or an ARB for treatment of cancer, in particular for treating bladder cancer, ovarian cancer, glioblastoma and other cancers such as melanoma and breast cancer.

### BACKGROUND OF THE INVENTION

Cancers continue to be among the most common and deadly disease. Elucidation of biochemical pathways involved in development and progression of various cancers is important to identify potential anti-cancer treatments as well as to develop agents effective to regulate such pathways in other aspects of health and disease.

Cancer remains a leading cause of death worldwide. The International Agency for Research on Cancer (IARC) estimates that in 2012 there were 14.1 million new cancer cases and 8.2 million cancer deaths worldwide. Further, the IARC estimates that by 2030, the global cancer burden is expected to grow to 21.7 million new cancer cases and 13 million cancer deaths simply due to the growth and aging of the population. In the U.S. alone, it is estimated that approximately 1,685,210 new cases of cancer will be diagnosed and almost 600,000 people will die from the disease. See, e.g., American Cancer Society, “Cancer Facts & Figures 2016,” Atlanta: American Cancer Society (2016).

Ovarian cancer is the ninth most common cancer in women and the fifth leading cause of cancer-related deaths in women in the U.S. One of every 72 women will develop ovarian cancer and one of every 100 will die from this form of cancer. The American Cancer Society estimates that in 2016, 22,280 women will be diagnosed with ovarian cancer and about 14,240 will die from ovarian cancer. About 85% to 90% of ovarian cancers are epithelial ovarian carcinomas. This high mortality rate reflects, in part, a lack of early symptoms and a lack of effective screening tests. Thus, ovarian cancer often is diagnosed at an advanced stage, after the disease has spread beyond the ovary. Thus, early diagnosis is key to enhancing treatment and reducing mortality.

Glioblastomas are tumors that arise from astrocytes, which are star-shaped cells that make up the supportive tissue of the brain. Glioblastoma (GBM) is the most common and most aggressive cancer that begins in the brain. The National Cancer Institute estimates that more than 23,000 new cases of brain cancer are predicted in the United States in 2013, with more than 14,000 people likely to die from the disease. Most patients with GBM die of the disease within approximately 15 months of diagnosis. Considering the rapid rate of progression, GBM is often diagnosed due to symptoms indicating increased pressure experience by the surrounding brain tissue. However, GBM cells are very infiltrative and, thus, can spread quickly into other parts of the brain, complicating treatment and prognosis.

Bladder cancer is any type of cancer arising from the epithelial lining of the urinary bladder. Bladder cancer is the ninth leading type of cancer. In 2010, bladder cancer resulted in 170,000 deaths worldwide, which is an increase from 114,000 deaths in 1990. The American Cancer Society estimates that in 2016, about 76,960 new cases of bladder cancer will be diagnosed in the U.S. and about 16,390 deaths will result from bladder cancer. While several risk factors exist for bladder cancer, there is no standard or routine screening test for bladder cancer. Often, cystoscopy, an invasive procedure to view the interior of the bladder and urethra is used if other factors suggest bladder cancer. Additionally, urine can be monitored for the presence of abnormal cells. This approach requires visual analysis of biological samples.

Significant research has been dedicated to identify novel drug targets for cancer prevention and treatment. Non-steroidal anti-inflammatory drugs (NSAIDs) are one group of compounds that have been found to decrease the risk of colorectal cancer. NSAIDs target and inhibit the cyclooxgenase enzymes, COX-1 and COX-2. Because elevated COX-2 expression has been found in approximately 50% of colorectal adenomas and 85% of colorectal adenocarcinomas, it has been hypothesised that NSAIDs may exert some of their anti-inflammatory and anti-tumour effects through inhibition of COX-2. Following this hypothesis, and the fact that many of the unwanted gastrointestinal side effects associated with NSAIDs are related to COX-1 inhibition, there has been a focus on the use of COX-2 selective NSAIDs for the treatment and prevention of colorectal cancer.

Celecoxib is a cyclooxygenase-2 inhibitor (COX-2) inhibitor and is routinely administered in the treatment of osteoarthritis, adult rheumatoid arthritis, and ankylosing spondylitis. As noted above, celecoxib has also demonstrated chemopreventative activity in colon carcinogenesis.

Olmesartan is an anti-hypertensive agent that is commonly administered to treat high blood pressure and diseases and disorders associated with high blood pressure. Unlike celecoxib, olmesartan is not known to have chemopreventative or chemotherapeutic properties.

The development of safe and effective cancer therapeutic agents continues to be significant medical effort. Furthermore, there remains a need for improved methods and reagents for treating bladder cancer, ovarian cancer, glioblastoma and other cancers such as melanoma and breast cancer. Despite these efforts, a need exists for new chemotherapeutic agents and cancer treatments that do not have the drawbacks associated with conventional chemotherapeutic agents. The present invention seeks to fulfill this need and provides further related advantages.

### SUMMARY OF THE INVENTION

In one aspect, the invention provides a method of treating cancer, comprising administering a therapeutically effective amount of olmesartan or a pharmaceutically acceptable salt thereof, and celecoxib or a pharmaceutically acceptable salt thereof, to a subject in need thereof. In another aspect, the invention provides a method of treating breast cancer, comprising administering a therapeutically effective amount of olmesartan or a pharmaceutically acceptable salt thereof, and celecoxib or a pharmaceutically acceptable salt thereof, to a subject in need thereof. In another aspect, the invention provides a method of treating skin cancer, comprising administering a therapeutically effective amount of olmesartan or a pharmaceutically acceptable salt thereof, and celecoxib or a pharmaceutically acceptable salt thereof, to a subject in need thereof. In another aspect, the invention provides a method of treating glioblastoma, comprising administering a therapeutically effective amount of olmesartan or a pharmaceutically acceptable salt thereof, and celecoxib or a pharmaceutically acceptable salt thereof, to a subject in need thereof.

In one embodiment, the invention provides a method of treating a cancer characterized by abnormal activation of COX-2, comprising administering a therapeutically effective amount of olmesartan or a pharmaceutically acceptable salt thereof, and celecoxib or a pharmaceutically acceptable salt thereof, to a subject in need thereof.

In one embodiment, the olmesartan and celecoxib are administered concurrently as a pharmaceutical composition. In one embodiment, administering the therapeutically effective amount of olmesartan and celecoxib comprises administering a fixed dose combination comprising olmesartan and celecoxib. In one embodiment, olmesartan and celecoxib are administered orally. In one embodiment, the subject is human. In one embodiment, the cancer is a breast cancer or a skin cancer, or a glioblastoma.

In one embodiment, the invention includes an assay of COX-2 to detect abnormal activation of COX-2 in a sample obtained from the subject containing cancer cells. In one embodiment the invention includes assay of angiotensin II receptor to detect abnormal level of angiotensin II receptor in a sample obtained from the subject containing cancer cells.

In one embodiment, the disease or condition is a proliferative disease or condition (e.g., cancer, restenosis, fibrosis)

The present invention provides, in one aspect, a method of modulating vascular resistance in a cancer subject in need thereof, the method comprising the step of administering to the subject a therapeutically effective amount of an angiotensin receptor blocker (ARB) or an angiotensin converting enzyme (ACE) inhibitor, thereby reducing tumor growth in the subject in need thereof.

In one embodiment, the ACE inhibitor is selected from the group consisting of benazepril, captopril, enalapril, fosinopril, moexipril, perindopril, quinapril, ramipril, trandolapril, and lisinopril. In one embodiment, the ACE inhibitor is Lisinopril.

In one embodiment, the ARB is selected from the group consisting of eprosartan, azilsartan medoxomil, valsartan, telmisartan, losartan, candesartan, irbesartan, and olmesartan. In one embodiment, the ARB is olmesartan.

In one embodiment, the cancer is selected from the group consisting of melanoma, glioblastoma, ovarian cancer, bladder cancer, and breast cancer. In one embodiment, the cancer is melanoma. In one embodiment, the cancer is glioblastoma. In one embodiment, the cancer is ovarian cancer. In one embodiment, the cancer is bladder cancer. In one embodiment, the cancer is breast cancer.

In one embodiment, the ACE inhibitor or ARB is administered concurrently with chemotherapeutic, hormonal, surgical, or radiation treatment.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 compares tumor volume (MDA-MB-435 xenograft) as a function of time and administration of celecoxib (CELEBREX®), olmesartan, and a combination of celecoxib and lisinopril (CELEBREX®+Lisinopril) versus control (saline).

FIG. 2 compares tumor volume (MDA-MB-435 xenograft) as a function of time and administration of celecoxib (CELEBREX®), olmesartan, and a combination of celecoxib and celecoxib and hydrochlorothiazide (CELEBREX®+HCTZ) versus control (saline).

FIG. 3 compares tumor volume (MDA-MB-435 xenograft) as a function of time and administration of celecoxib (CELEBREX®), olmesartan, and a combination of celecoxib and celecoxib and olmesartan (CELEBREX®+Olmesartan) versus control (saline).

FIG. 4 shows a comparison of SUM149-RR tumor growth when treated with CEL and anti-HTNs as a single agent and in combination.

FIG. 5 shows suppression of lymph node metastasis in SUM149-RR.

FIG. 6 shows the effect of CEL and three antihypertensive drugs as single agents or in combination on ILN and lung metastasis. Low blood vessel (BV) density and necrosis was apparent in control tumors (left top panel). In contrast, OLM facilitated the normalization and stabilization of tumor BVs (left bottom panels). LIS had no major effect. Recruitment of CD11 b monocytes also correlated with higher number of BV in tumors (right panels).

FIG. 7 shows non age-adjusted BNP levels in patients with bladder, brain, ovarian and pancreatic cancers versus donor group.

FIG. 8 shows age-adjusted BNP levels in patients with bladder, brain, ovarian and pancreatic cancers versus donor group.

FIG. 9 shows the effect of 3 anti-HTN drugs on lymph node metastasis.

FIG. 10 shows the effect of 3 anti-HTN drugs on lung metastasis.

### DETAILED DESCRIPTION OF THE INVENTION

A significant reduction in age-adjusted BNP levels was previously demonstrated in patients with glioblastoma, bladder and ovarian cancers but not pancreatic cancer (see U.S. Provisional Patent Application Nos. 62/316,318 and 62/316,346, herein incorporated by reference in their entirety). Natriuretic peptides are peptide hormones that are synthesized by the heart, brain and other organs. The release of these peptides by the heart is stimulated by arterial and ventricular distension and by neurohumoral stimuli, usually in response to heart failure. The main physiological action of natriuretic peptides is to reduce arterial pressure by decreasing blood volume and systemic vascular resistance. Thus, in the glioblastoma, bladder cancer and ovarian cancer patients studied, arterial pressure is elevated, at least in part, due to a decrease in BNP levels.

To further study this relationship of vascular resistance and cancer, in the present application the inventors studied the anticancer effects of anti-hypertensive agents on tumor growth and metastasis in metastatic xenograft models. As shown below in the example section, three anti-hypertensive drugs (lisinopril [LIS], olmesartan medoxomil [OLM] and hydrochlorothiazide [HCTZ]) were evaluated in cancer models of breast cancer, melanoma, and glioblastoma cancer models. Lisinopril, an ACE inhibitor, and olmesartan, an ARB, showed tumor growth suppression in the cancer models studied. HTCZ, a diuretic that acts by decreasing the kidney's ability to retain water, did not show the same effect. Olmesartan and lisinopril lower the blood pressure by directly decreasing the vascular resistance unlike HCTZ, an antihypertensive diuretic which has no direct impact on the vascular wall.

Without being limited by theory, Applicants believe that the ACE inhibitors and ARB, exemplified by lisinopril and olmesartan, respectively, act to decrease vascular resistance and therefore reduce tumor growth. Consistent with this hypothesis, we predict that ACE inhibitors and ARB will act to reduce tumor growth in cancers that also show a reduction in BNP levels, such as bladder cancer, ovarian cancer, glioblastoma (but not pancreatic cancer), as well as in breast cancer and melanoma, which are shown here to be responsive to ACE inhibitors and ARB.

Angiotensin Converting Enzyme (ACE) inhibitors modulate angiotensin-converting enzyme, an important component of the renin-angiotensin-aldosterone system. ACE inhibitors block the conversion of angiotensin I (AI) to angiotensin II (AII). They thereby lower arteriolar resistance and increase venous capacity; lower resistance in blood vessels in the kidneys; and lead to increased natriuresis (excretion of sodium in the urine). ACE inhibitors include benazepril, captopril, enalapril, fosinopril, moexipril, perindopril, quinapril, ramipril, trandolapril, and lisinopril.

Angiotensin II receptor antagonists, also known as angiotensin receptor blockers (ARBs), AT1-receptor antagonists or sartans, are a group of pharmaceuticals that modulate the renin-angiotensin system. These substances are AT1-receptor antagonists; that is, they block the activation of angiotensin II AT1 receptors. Blockage of AT1 receptors directly causes vasodilation, reduces secretion of vasopressin, and reduces production and secretion of aldosterone, among other actions. The combined effect reduces blood pressure. ARBs include eprosartan, azilsarta, valsartan, telmisartan, losartan, candesartan, irbesartan, and olmesartan.

The compositions of the invention, either ACE inhibitors or ARBs, or a combination of both, are administered to subjects diagnosed with glioblastoma, bladder cancer, ovarian cancer, breast cancer and melanoma, alone, in combination with, before or after a conventional cancer treatment such as chemotherapy, hormonal therapy, radiation therapy, and surgery.

In another aspect, the present invention provides also methods and compositions for treating cancers, such as breast cancer and skin cancer, using a combination of celecoxib and olmesartan. In another aspect, the invention provides a composition that includes celecoxib and olmesartan that is useful for treating cancers, such breast cancer or skin cancer. In a further aspect, the invention provides a commercial package that includes a combination of celecoxib and olmesartan.

The present invention provides a novel combination of pharmacological drugs that inhibit inflammation and hypertension known to intensity inflammatory conditions as well as a novel formulation of an established anti-cancer chemotherapeutic, paclitaxel. The invention is based in part on a preclinical model of human melanoma in immunodeficient mice. Cyclooxygenase-2 (COX-2) is an inducible enzyme synthesizing prostaglandins such as PGD2 and PGE2 from arachidonic acid. COX-2 is upregulated in most human tumors and is a potent inducer of cancer-associated inflammation that promotes tumor angiogenesis and lymphangiogenesis thereby enhancing hematogenous and lymphatic metastasis. Celecoxib is a well-established specific inhibitor of COX-2 with known anti-tumor effects. Hypertension is recently emerged as a causal factor for tumor progression and anti-hypertension agents have been to reduce inflammation and suppress tumor growth and metastasis. Three anti-hypertensive drugs are evaluated:

(1) Lisinopril, an inhibitor of angiotensin-1 converting enzyme (ACE); (2) olmesartan, an angiotensin II receptor blocker, ARB; and (3) hydrochlorothiazide (HCTZ). First, the anti-tumor growth and anti-metastatic will be assessed for efficacy of celecoxib, lisinopril, ARB, and HCTZ used as monotherapies or the latter three drugs in combination with celecoxib.

It has been surprisingly found that only the combination of celecoxib and olmesartan is effective for reducing tumor growth in a xenograft model (MDA-MB-435, breast and skin cancer cell line). Combinations of celecoxib with lisinopril or HCTZ were ineffective.

Methods of Treatment

In one aspect, the invention provides methods for the treatment of cancer, such as breast cancer or skin cancer or glioblastoma. In the methods, a therapeutically effective amount of a combination of celecoxib and olmesartan is administered to a subject in need thereof.

In certain embodiments, the invention provides a method of treating breast cancer. In one embodiment, the method comprises administering a therapeutically effective amount of olmesartan or a pharmaceutically acceptable salt thereof, and celecoxib or a pharmaceutically acceptable salt thereof, to a subject in need thereof.

In other embodiments, the invention provides a method of treating glioblastoma. In one embodiment, the method comprises administering a therapeutically effective amount of olmesartan or a pharmaceutically acceptable salt thereof, and celecoxib or a pharmaceutically acceptable salt thereof, to a subject in need thereof.

In other embodiments, the invention provides a method of treating skin cancer. In one embodiment, the method comprises administering a therapeutically effective amount of olmesartan or a pharmaceutically acceptable salt thereof, and celecoxib or a pharmaceutically acceptable salt thereof, to a subject in need thereof.

In further embodiments, the invention provides a method of treating a cancer characterized by abnormal activation of COX-2. In one embodiment, the method comprises administering a therapeutically effective amount of olmesartan or a pharmaceutically acceptable salt thereof, and celecoxib or a pharmaceutically acceptable salt thereof, to a subject in need thereof.

In certain embodiments of the above methods, the ACE inhibitors, ARB, or olmesartan and celecoxib are administered concurrently as a pharmaceutical composition. In certain embodiments, olmesartan and celecoxib are administered as a fixed dose combination. In certain embodiments, the ACE inhibitors, ARB, or olmesartan and celecoxib are administered orally.

The terms “treating” and “treatment” used to refer to treatment of breast cancer, skin cancer and glioblastoma in a subject include preventing, inhibiting or ameliorating the cancer in the subject, such as slowing progression of the cancer and/or reducing or ameliorating a sign or symptom of the cancer.

A therapeutically effective amount of an ACE inhibitor, ARB, or celecoxib and olmesartan administered according to aspects of the present invention is an amount which has a beneficial effect in a subject being treated. In subjects having cancer or at risk for having cancer, such as a condition characterized by abnormal cell proliferation including, but not limited to, pre-neoplastic hyperproliferation, cancer in-situ, neoplasms, metastasis, a tumor, a benign growth or other condition responsive to an inventive composition, a therapeutically effective amount of a composition is effective to ameliorate or prevent one or more signs and/or symptoms of the condition. For example, a therapeutically effective amount of a composition including celecoxib and olmesartan is effective to detectably increase apoptosis and/or decrease proliferation of cells of a cancer condition characterized by abnormal cell proliferation including, but not limited to, pre-neoplastic hyperproliferation, cancer in-situ, neoplasms, metastasis, a tumor, a benign growth or other condition responsive to an inventive composition.

Methods of treating a subject are provided according to aspects of the present invention which include administering a therapeutically effective amount of the ACE inhibitors, ARB, or celecoxib and olmesartan to a subject in need thereof, wherein the subject has an abnormal proliferative condition, such as cancer, pre-neoplastic hyperproliferation, cancer in-situ, neoplasms, metastasis, tumor or benign growth.

Subjects are identified as having, or at risk of having, cancer using well-known medical and diagnostic techniques. The term “subject” refers to an individual in need of treatment for a pathological condition responsive to the beneficial effects of compositions of the present invention, particularly breast or skin cancers. While the present invention describes compositions and methods for treatment of human subjects in need thereof, the present invention is not limited to human subjects and the term subject generally includes mammals.

A therapeutically effective amount of an ACE inhibitor, ARB, or celecoxib and olmesartan according to the present invention will vary depending on the severity of the condition to be treated, the species of the subject, the age and sex of the subject, and the general physical characteristics of the subject to be treated. One of skill in the art could determine a therapeutically effective amount in view of these and other considerations typical in medical practice. In general, it is contemplated that a therapeutically effective amount would be in the range of about 0.001 mg/kg-100 mg/kg body weight, optionally in the range of about 0.01-10 mg/kg, and further optionally in the range of about 0.1-5 mg/kg. Further, dosage may be adjusted depending on whether treatment is to be acute or continuing.

Combination Treatments

Combinations of therapeutic agents are administered according to aspects of the present invention. In some aspects, an ACE inhibitor, ARB, or celecoxib and olmesartan, and at least one additional therapeutic agent are administered to a subject to treat cancer in a subject in need thereof. The term “additional therapeutic agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues which is a biologically, physiologically, or pharmacologically active substance (or substances) that acts locally or systemically in a subject.

Additional therapeutic agents included in aspects of methods and compositions of the present invention include, but are not limited to, antibiotics, antivirals, antineoplastic agents, analgesics, antipyretics, antidepressants, antipsychotics, anti-cancer agents, antihistamines, anti-osteoporosis agents, anti-osteonecrosis agents, anti-inflammatory agents, anxiolytics, chemotherapeutic agents, diuretics, growth factors, hormones, non-steroidal anti-inflammatory agents, steroids and vasoactive agents.

Treatments including administration of both celecoxib and olmesartan show synergistic effects. Combination therapies utilizing celecoxib, olmesartan, and one or more additional therapeutic agents may show further synergistic effects. According to aspects of the present invention, combination therapies include: (1) administration of pharmaceutical compositions that include celecoxib and olmesartan of the present invention in combination with one or more additional therapeutic agents; (2) co-administration of celecoxib and olmesartan with one or more additional therapeutic agents wherein celecoxib and olmesartan are formulated in the same composition and wherein the one or more additional therapeutic agents have not been formulated in the same composition. When using separate formulations, celecoxib, olmesartan, and the one or more additional therapeutic agents may be administered at the same time or at different times; and two or more of celecoxib, olmesartan and the one or more additional therapeutic agents may be administered at the same time or at different times with reference to the other therapeutic agents.

Combination treatments including the ACE inhibitors, ARB, or celecoxib and olmesartan with one or more additional therapeutic agents can allow for reduced effective dosage and increased therapeutic index of the compositions of the present invention and the one or more additional therapeutic agents used in methods of the present invention.

Optionally, a method of treating a subject having cancer or at risk of having cancer further includes an adjunct anti-cancer treatment. An adjunct anti-cancer treatment can be administration of an anti-cancer agent. Anti-cancer agents are described, for example, in Goodman et al., Goodman and Gilman's The Pharmacological Basis of Therapeutics, 8th Ed., Macmillan Publishing Co., 1990. Anti-cancer agents illustratively include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflornithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochloride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin.

Additional agents include immunostimulating agents, such as INF, IL-15, NK, CART, PD-1, PDL-1, and TGF-beta inhibitor.

An adjunct anti-cancer treatment can be a radiation treatment of a subject or an affected area of a subject's body. In particular aspects, cancers treated in a subject using methods and compositions described herein are characterized by abnormal activation of COX-2 or abnormal angiotensin II receptor levels. Increased levels or activity of COX-2 can be determine by measurement of gene copy number, protein or RNA levels in cells known or suspected to be dysplasic, pre-cancerous, cancerous, metastatic or otherwise characterized by abnormal cell proliferation compared to normal cells. Assays for abnormal activation of COX-2 include phosphorylation assays, immunoassays and nucleic acid assays.

Pharmaceutical Compositions

In another aspect, the present invention provides compositions comprising an ACE inhibitor, ARB, or celecoxib and olmesartan useful for the treatment of cancers, such as breast cancer, skin cancer, and glioblastoma.

Celecoxib, 4-[5-(4-methylphenyl)-3-(trifluoromethyl)-1H-pyrazol-1-yl]benzenesulfonamide, is a cyclooxygenase-2 (COX-2) inhibitor. Celecoxib can be obtained commercially or chemically synthesized according to known methods.

Olmesartan, (5-methyl-2-oxo-2H-1,3-dioxol-4-yl)methyl 4-(2-hydroxypropan-2-yl)-2-propyl-1-({4-[2-(2H-1,2,3,4-tetrazol-5-yl)phenyl]phenyl}methyl)-1H-imidazole-5-carboxylate, (e.g., olmesartan medoxomil) is an angiotensin II receptor antagonist that has been used for the treatment of high blood pressure. Olmesartan can be obtained commercially or chemically synthesized according to known methods.

Compositions including celecoxib may be provided as a pharmaceutically acceptable salt of celecoxib. Compositions including olmesartan may be provided as a pharmaceutically acceptable salt of olmesartan. Compositions including an ACE inhibitor or an ARB may be provided as a pharmaceutically acceptable salt. A “pharmaceutically acceptable” salt is suitable for use in a subject without undue toxicity or irritation to the subject and is effective for their intended use. Pharmaceutically acceptable salts include pharmaceutically acceptable acid addition salts and base addition salts. Pharmaceutically acceptable salts are well-known in the art, such as those detailed in S. M. Berge et al., J. Pharm. Sci., 66:1-19, 1977. Exemplary pharmaceutically acceptable salts are those suitable for use in a subject without undue toxicity or irritation to the subject and which are effective for their intended use which are formed with inorganic acids such as hydrochloric acid, hydrobromic acid, hydroiodic acid, nitric acid, phosphoric acid, sulfuric acid and sulfamic acid; organic acids such as acetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, formic acid, fumaric acid, glutamic acid, glycolic acid, glycerophosphoric acid, hemisulfic acid, heptanoic acid, hexanoic acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, trichloroacetic acid, trifluoroacetic acid and undecanoic acid; inorganic bases such as ammonia, hydroxide, carbonate, and bicarbonate of ammonium; organic bases such as primary, secondary, tertiary and quaternary amine compounds ammonium, arginine, betaine, choline, caffeine, diolamine, diethylamine, diethanolamine, 2-dimethylaminoethanol, 2-diethylaminoethanol, dicyclohexylamine, dicyclohexylamine, dibenzylamine, N,N-dibenzylphenethylamine, 1-ephenamine, N,N′-dibenzylethylenediamine, ethanolamine, ethylamine, ethylenediamine, glucosamine, histidine, hydrabamine, isopropylamine, lysine, methylamine, N-ethylpiperidine, N-methylpiperidine, N-methylmorpholine, N,N-dimethylaniline, piperazine, trolamine, methylglucamine, purines, piperidine, pyridine, theobromine, tetramethylammonium compounds, tetraethylammonium compounds, trimethylamine, triethylamine, tripropylamine and tributylamine and metal cations such as aluminum, calcium, copper, iron, lithium, magnesium, manganese, potassium, sodium, and zinc.

In certain aspects, the composition is a pharmaceutical composition that includes an ACE inhibitor, ARB, or celecoxib and olmesartan, and a pharmaceutically acceptable carrier. The term “pharmaceutically acceptable carrier” refers to a carrier which is substantially non-toxic to a subject to which the composition is administered and which is substantially chemically inert with respect to the active component or components.

Pharmaceutically acceptable carriers and formulation of pharmaceutical compositions are known in the art, such as described in Remington: The Science and Practice of Pharmacy, 21^{st} Ed., Lippincott, Williams & Wilkins, Philadelphia, Pa., 2006; and Allen, L. V. et al., Ansel's Pharmaceutical Dosage Forms and Drug Delivery Systems, 8^{th} Ed., Lippincott, Williams & Wilkins, Philadelphia, Pa., 2005. A pharmaceutical composition according to the invention generally includes about 0.1-99% of celecoxib and olmesartan. The pharmaceutical composition of the present invention is suitable for oral administration to a subject.

In some embodiments, the composition is suitable for administration to a human. In some embodiments, the composition is suitable for administration to a mammal, such as, in the veterinary context, including domestic pets and agricultural animals. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, (d) suitable emulsions, and (e) powders. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The combination of celecoxib and olmesartan can include a “fixed dose combination” (FDC). These fixed dose combinations can be in the form of pill in pill, capsule in capsule, bilayer tablet, a tablet in powder in a capsule, or a mini-tablet in powder in a capsule, or other formulation method with physical separation between celecoxib and olmesartan. The composition may be administered, for example, daily, twice a day, three times a day, four times a day, or every other day.

In some embodiments, the composition is formulated to have a pH in the range of about 4.5 to about 9.0, including for example pH in the ranges of any of about 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 7.5 or about 8). The composition can also be made to be isotonic with blood by the addition of a suitable tonicity modifier such as glycerol.

Articles of Manufacture

In further aspects, the invention provides articles of manufacture comprising the compositions described herein in suitable packaging. Suitable packaging for compositions described herein are known in the art, and include, for example, vials (such as sealed vials), vessels (such as sealed vessels), ampules, bottles, jars, flexible packaging (such as sealed Mylar or plastic bags), and the like. These articles of manufacture may further be sterilized and/or sealed.

Also provided are unit dosage forms comprising the compositions described herein. These unit dosage forms can be stored in a suitable packaging in single or multiple unit dosages and may also be further sterilized and sealed.

The present invention also provides kits comprising compositions (or unit dosages forms and/or articles of manufacture) described herein and may further comprise instruction(s) on methods of using the composition, such as uses further described herein. In some embodiments, the kit of the invention comprises the packaging described above. In other embodiments, the kit of the invention comprises the packaging described above and a second packaging comprising a buffer. It may further include other materials desirable from a commercial and user standpoint, including other buffers, diluents, filters, needles, syringes, and package inserts with instructions for performing any methods described herein.

Kits may also be provided that contain sufficient dosages of the therapeutic agent as disclosed herein to provide effective treatment for an individual for an extended period, such as any of a week, 2 weeks, 3 weeks, 4 weeks, 6 weeks, 8 weeks, 3 months, 4 months, 5 months, 6 months, 7 months, 8 months, 9 months or more. Kits may also include multiple unit doses of the therapeutic agent and pharmaceutical compositions and instructions for use and packaged in quantities sufficient for storage and use in pharmacies, for example, hospital pharmacies and compounding pharmacies.

Methods of Using the Compositions

The term “effective amount” used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to cause a tumor to shrink and/or to decrease the growth rate of the tumor (such as to suppress tumor growth). In some embodiments, an effective amount is an amount sufficient to delay development. In some embodiments, an effective amount is an amount sufficient to prevent occurrence and/or recurrence. An effective amount can be administered in one or more administrations.

The compositions of the invention are effective for treating proliferative diseases including cancers, restenosis, and fibrosis, among others

Cancers to be treated by compositions described herein include, but are not limited to, carcinoma, lymphoma, glioblastoma, sarcoma, and leukemia. Examples of cancers that can be treated by compositions described herein include, but are not limited to, squamous cell cancer, lung cancer (including small cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, melanoma, endometrial or uterine carcinoma, salivary gland carcinoma, kidney or renal cancer, liver cancer, prostate cancer, vulval cancer, thyroid cancer, hepatic carcinoma, head and neck, cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), myeloma, Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome. In some embodiments, there is provided a method of treating metastatic cancer (that is, cancer that has metastasized from the primary tumor). In some embodiments, there is provided a method of reducing cell proliferation and/or cell migration. In some embodiments, there is provided a method of treating hyperplasia.

In some embodiments, there are provided methods of treating cancer at advanced stage(s). In some embodiments, there are provided methods of treating breast cancer (which may be HER2 positive or HER2 negative), including, for example, advanced breast cancer, stage IV breast cancer, locally advanced breast cancer, and metastatic breast cancer. In some embodiments, the cancer is lung cancer, including, for example, non-small cell lung cancer (NSCLC, such as advanced NSCLC), small cell lung cancer (SCLC, such as advanced SCLC), and advanced solid tumor malignancy in the lung. In some embodiments, the cancer is ovarian cancer, head and neck cancer, gastric malignancies, melanoma (including metastatic melanoma), colorectal cancer, pancreatic cancer, and solid tumors (such as advanced solid tumors). In some embodiments, the cancer is any of (and in some embodiments selected from the group consisting of) breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, gliomas, glioblastomas, neuroblastomas, and multiple myeloma. In some embodiments, the cancer is a solid tumor. In some embodiments, the cancer is any of (in some embodiments, selected from the group consisting of) prostate cancer, colon cancer, breast cancer, head and neck cancer, pancreatic cancer, lung cancer, and ovarian cancer.

Individuals suitable for receiving these compositions depend on the nature of the therapeutic agent, as well as the disease/condition/disorder to be treated and/or prevented. Accordingly, the terms “individual” and “subject” include any of vertebrates, mammals, and humans depending on intended suitable use. In some embodiments, the individual is a mammal. In some embodiments, the individual is any one or more of human, bovine, equine, feline, canine, rodent, or primate. In some embodiments, the individual is a human.

In further embodiments, the invention provides a method of treating carcinoma (such as colon carcinoma) in an individual, wherein the method comprises administering to the individual a composition comprising an effective amount of therapeutic agent.

The compositions described herein can be administered alone or in combination with other pharmaceutical agents, such as described above.

The dose of the composition of the invention administered to an individual will vary with the particular composition, the method of administration, and the particular disease being treated. The dose is sufficient to effect a desirable response, such as a therapeutic or prophylactic response against a particular disease or condition. For example, the dosage of representative therapeutic agents (e.g., paclitaxel) administered can be about 1 to about 300 mg/m², including for example about 10 to about 300 mg/m², about 30 to about 200 mg/m², and about 70 to about 150 mg/m². Typically, the dosage of a therapeutic agent (e.g., paclitaxel) in the composition can be in the range of about 50 to about 200 mg/m² when given on a 3 week schedule, or about 10 to about 100 mg/m² when given on a weekly schedule. In addition, if given in a metronomic regimen (e.g., daily or a few times per week), the dosage may be in the range of about 1-50 mg/m².

Dosing frequency for the compositions of the invention includes, but is not limited to, at least about any of once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequent.

The administration of the compositions of the invention can be extended over an extended period of time, such as from about a month up to about three years. For example, the dosing can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

The compositions described herein can be administered to an individual via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In certain embodiments, the compositions are administered by any acceptable route including, but not limited to, orally, intramuscularly, transdermally, and intravenously.

When preparing the compositions for injection, particularly for intravenous delivery, the continuous phase preferably comprises an aqueous solution of tonicity modifiers, buffered to a pH range of about 5 to about 8.5. The pH may also be below 7 or below 6. In some embodiments, the pH of the composition is no less than about 6, including for example no less than about any of 6.5, 7, or 8 (such as about 7.5 or 8).

The therapeutic agents useful in the invention can be enclosed in a hard or soft capsule, can be compressed into tablets, or can be incorporated with beverages or food or otherwise incorporated into the diet. Capsules can be formulated by mixing the nanoparticles with an inert pharmaceutical diluent and inserting the mixture into a hard gelatin capsule of the appropriate size. If soft capsules are desired, a slurry of the nanoparticles with an acceptable vegetable oil, light petroleum or other inert oil can be encapsulated by machine into a gelatin capsule.

The methods of the invention include methods of making pharmaceutical compositions comprising combining any of the compositions described herein with a pharmaceutically acceptable excipient.

In a further aspect, the invention provides use of the compositions described herein in the manufacture of a medicament. Particularly, the manufacture of a medicament for use in the treatment of conditions described herein. Further, the pharmaceutical composition thereof described herein, are also intended for use in the manufacture of a medicament for use in treatment of the conditions and, in accordance with the methods, described herein.

In certain embodiments, the present invention provides fixed dose combinations that include celecoxib and olmesartan.

In certain embodiments, the methods of the invention, celecoxib and olmesartan are administered as a fixed dose combination (FDC). As used herein, the term “fixed dose combination” or “FDC” refers to a combination of two therapeutic agents (e.g., an antihypertensive drug and an analgesic agent) formulated in a single composition in which the amount of each is fixed in the dosage form (e.g., tablet).

The fixed dose combination can be in the form of a capsule or tablet (e.g., a bilayered tablet). In certain embodiments, the FDC has an enteric coating.

In certain embodiments, the fixed dose combination comprises celecoxib and olmesartan in a weight/weight ratio of from 20:1 to 4:1.

In certain embodiments, the fixed dose combination comprises 50 to 800 mg of celecoxib, and 2.5 to 100 mg of olmesartan.

An improved pharmaceutical composition that is a single dosage form (e.g. FDC) of olmesartan medoxomil) and celecoxib) is described in WO 2015/191473, expressly incorporated herein by reference in its entirety. This single dosage form comprises separate compartments for each drug in which each drug is separately and independently formulated. When the single dosage form is administered the interaction to in vivo absorption is minimized and the combination formulation is bioequivalent to the single formulation of each of drugs.

In certain embodiments, the subject treatable by the methods of the invention is a subject that is in need of treatment for breast cancer and melanoma. In certain embodiments of this method, a single dosage form that comprises olmesartan and celecoxib is administered.

A description of representative single dosage forms useful in the methods of the invention and methods for making the single dose forms are described in WO 2015/191473, expressly incorporated herein by reference in its entirety. Representative single dose forms useful in the method of the inventions and a method for making them are described below.

The pharmaceutical composition useful in the methods of the invention, which includes olmesartan medoxomil and celecoxib, are formulated into a combination dosage form having separate compartments. That is, the pharmaceutical composition has a single dosage form comprising a compartment comprising olmesartan medoxomil; and a compartment comprising celecoxib, wherein the compartments are formulated in a separate form.

In the pharmaceutical composition, the active ingredients (i.e., an ACE inhibitor, ARB, or olmesartan medoxomil and celecoxib) may be used in a therapeutically effect amount. For example, olmesartan medoxomil may be used in an amount of about 5 mg to about 80 mg, preferably about 10 mg to about 40 mg, in a unit formulation (i.e., unit dosage form).; and celecoxib may be used in an amount of about 2 mg to about 40 mg, preferably about 5 mg to about 20 mg, in a unit formulation (i.e., unit dosage form). The pharmaceutical composition may be administered once a day, but not limited thereto.

The pharmaceutical composition has a combination dosage form having separate compartments (i.e., a double-layered tablet form), comprising or consisting essentially of a layer comprising celecoxib and a layer comprising olmesartan medoxomil.

When the compartment comprising celecoxib includes a certain disintegrant (i.e., cellulose-type and/or povidone-type disintegrants), in a certain amount, rapid disintegration and high initial dissolution rate of celecoxib can be accomplished, thereby being able to obtain a combination formulation bioequivalent to the single formulation of celecoxib. The disintegrant may be one or more selected from the group consisting of povidone (for example, KOLIDONE™), crospovidone (for example, POLYPLASDONE™), low substituted hydroxypropyl cellulose, croscarmellose sodium, and carboxymethylcellulose calcium. Preferably, the disintegrant may be a mixture of crospovidone and croscarmellose sodium; or croscarmellose sodium. The disintegrant may be present in an amount ranging from 2 to 20% by weight, preferably from 3 to 15% by weight, based on the total weight of the compartment comprising celecoxib. When other disintegrants are used, the dissolution rate of rosuvastatin or its salt is decreased; and/or the amount used is increased, which may cause insufficient compression force during the compressing step, thereby leading to high friability of the resulting formulation (e.g., tablet). In addition, the use of other disintegrants brings about insufficient hardness, which may cause unwanted problems in packaging or delivery.

For olmesartan medoxomil, a combination formulation comprising celecoxib and olmesartan medoxomil should be designed so as to exhibit high dissolution rate of olmesartan medoxomil in an in vitro comparative dissolution test, in order to obtain a bioequivalent formulation to the single formulation containing olmesartan medoxomil. In order to obtain the high in vitro dissolution rate, the compartment comprising olmesartan medoxomil comprises a preferred disintegrant, which may be one or more selected from the group consisting of low substituted hydroxypropyl cellulose, carboxymethylcellulose calcium, croscarmellose sodium, crospovidone, sodium starch glycolate, and pregelatinized starch. In an embodiment, the compartment comprising olmesartan medoxomil comprises 7.5 or more % by weight of low substituted hydroxypropyl cellulose, 5 or more % by weight of carboxymethylcellulose calcium, 15 or more % by weight of croscarmellose sodium, 10 or more % by weight of crospovidone, 5 or more % by weight of sodium starch glycolate, or 5 or more % by weight of pregelatinized starch, based on the total weight of the compartment comprising olmesartan medoxomil. In another embodiment, the compartment comprising olmesartan medoxomil comprises 7.5 to 65% by weight of low substituted hydroxypropyl cellulose, 5 to 60% by weight of carboxymethylcellulose calcium, 15 to 30% by weight of croscarmellose sodium, 10 to 40% by weight of crospovidone, 5 to 40% by weight of sodium starch glycolate, or 5 to 60% by weight of pregelatinized starch, based on the total weight of the compartment comprising olmesartan medoxomil. In a further embodiment, the compartment comprising olmesartan medoxomil comprises 7.5 to 65% by weight, preferably 10 to 60% by weight, more preferably about 20±1% by weight of low substituted hydroxypropyl cellulose, based on the total weight of the compartment comprising olmesartan medoxomil.

The pharmaceutical composition may further comprise one or more excipients conventionally used in the field of pharmaceutics, for example a diluent (or additive), a binder, a lubricant, in addition to said disintegrant. The pharmaceutical composition may be also coated with an appropriate coating agent, such as a film-coating agent.

The diluent (or additive) includes lactose (including its hydrate), dextrin, mannitol, sorbitol, starch, microcrystalline cellulose, silicified microcrystalline cellulose, calcium hydrogen phosphate (including its hydrate), anhydrous calcium hydrogen phosphate, calcium carbonate, saccharides, and a mixture thereof. The binder includes polyvinylpyrrolidone, copovidone, gelatin, starch, sucrose, methyl cellulose, ethyl cellulose, hydroxyethyl cellulose, hydroxypropyl cellulose, hydroxypropyl alkylcellulose (for example, hydroxypropyl methylcellulose), and a mixture thereof. The lubricant includes stearic acid, stearates (for example, magnesium stearate), talc, corn starch, carnauba wax, light anhydrous silicic acid, magnesium silicate, synthetic aluminum silicate, hydrogenated oil, hydrogenated oil, titanium oxide, microcrystalline cellulose, macrogol 4000 or 6000, isopropyl myristate, calcium hydrogen phosphate, and a mixture thereof. The coating agent, for example a film-coating agent, includes a conventional polymer. The film-coating agent may be used in a minimum amount for providing an appropriate size of the formulation, but not limited thereto.

The pharmaceutical composition having a double-layered tablet form may be prepared by preparing granules containing celecoxib and granules containing olmesartan medoxomil, respectively; and then compressing the mixture thereof with a double-layer tablet-press machine. If necessary, the resulting double-layered tablet may be coated with a film-coating agent such as OPADRY®. The granules containing rosuvastatin and the granules containing olmesartan medoxomil may be prepared according to dry granulation methods or wet granulation methods. For example, the granules containing celecoxib may be prepared according to a dry granulation method. That is, the granules containing celecoxib may be prepared by mixing rosuvastatin calcium, an additive (diluent), a disintegrant, and a lubricant according to a conventional method; and then granulating the mixture with, e.g., a roller compactor (TF mini, Vector). And also, the granules containing olmesartan medoxomil may be prepared according to a wet granulation method. That is, the granules containing olmesartan medoxomil may be prepared by mixing olmesartan medoxomil, a binder, an additive (diluent), a disintegrant; granulating the mixture with a high speed mixer (MIC Developer-5, COMASA); and then drying and sieving the resulting granules.

Representative double-layer tablets can be prepared as described below.

Step 1. Preparation of Granules Containing Celecoxib.

Celecoxib, lactose monohydrate, PROSOLV®, dibasic calcium phosphate dihydrate, crospovidone, croscarmellose sodium, light anhydrous silicic acid, and magnesium stearate (85% of the total amount used in the rosuvastatin-layer) were sieved through a 24 mesh and then mixed. The resulting mixture was granulated using a roller compactor (TF mini, Vector). The obtained granules were sieved through a 24 mesh and then mixed with magnesium stearate pre-sieved though a 35 mesh (15% of the total amount used in the rosuvastatin-layer) to prepare a celecoxib-containing granule mixture.

Step 2. Preparation of Granules Containing Olmesartan Medoxomil.

Olmesartan medoxomil, hydroxypropyl cellulose, lactose monohydrate, microcrystalline cellulose, and low substituted hydroxypropyl cellulose were sieved through a 24 mesh and then mixed. The resulting mixture was granulated using a high speed mixer (MIC Developer-5, COMASA). The resulting dry granules were sieved through a 24 mesh and then mixed with magnesium stearate pre-sieved though a 35 mesh and yellow iron oxide pre-sieved through a 80 mesh to prepare a olmesartan medoxomil-containing granule mixture.

Step 3. Preparation of Double-Layered Tablets.

The celecoxib-containing granule mixture prepared in Step 1 and the olmesartan medoxomil-containing granule mixture prepared in Step 2 were compressed with a double-layer tablet-press machine (BB-11, RIVA) to obtain double-layered tablets. The resulting tablets were film-coated with OPADRY® in a pan coating machine (LDCS, VECTOR).

While the preferred embodiment of the invention has been illustrated and described, it will be appreciated that various changes can be made therein without departing from the spirit and scope of the invention.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, pharmaceutical compositions, or methods and uses disclosed herein.

### Example 1

Tumor Model.

An established breast/melanoma cancer line (MDA-MB-435) was used for tumor studies. The cell line was tagged with Firefly luciferase to accurately determine the incidence and the burden of locoregional and systemic spread. The model has been extensively studied and has been shown to spread to lymph nodes and lungs.

Mice.

Five to six-weeks old immunodeficient female CB-17 SCID mice (Harlan, Indianapolis, Ind.) were used. All experiments were done in compliance with the regulations of the Southern Illinois University School of Medicine (SIU-SOM). Mice were housed in specific pathogen-free environment with 12 hours light/dark cycles at the vivarium of SIU-SOM.

Study No. 1.

Mice were orthotopically implanted with 4×106 cancer cells suspended in 100 μl of sterile Dulbecco's phosphate buffered saline (DPBS). Mice were assigned into eight experimental groups initiated 7 days post-tumor inoculation with the day of injection assigned ad Day 0. The groups are as follows:

1. Group 1—Control, treated with 100 μl of endotoxin-free saline by oral gavage p.o.; n=10

2. Group 2—p.o. administration of Celebrex (25 mg/kg/d, qdx5, 2 cycles); n=10

3. Group 3—p.o. administration of an ACE inhibitor Lisinopril (15 mg/kg/d, qdx5, 2 cycles); n=10

4. Group 44—p.o. administration of an angiotensin receptor inhibitor, ARB (30 mg/kg/d, qdx5, 2 cycles); n=10

5. Group 5—p.o. administration of HCTZ (10 mg/kg/d, qdx5, 1 cycle); n=10

6. Group 6—p.o. administration of Celebrex combined with Lisinopril; n=10

7. Group 7—p.o. administration of Celebrex combined with ARB; n=10

8. Group 8—p.o. administration of Celebrex combined with HCTZ; n=10

Number of mice used for Study 1: 80 mice plus 10% for attrition 80+8=88 mice total.

### Tested/Reported Parameters for Study No. 1 and Study No. 2: Mice

were monitored twice a week for 16 weeks as follows:

1. Percent weight loss (weight measurement twice/week, % weight change from day 0 vs. Days post-treatment).

2. Tumor volume measured by digital calipers and survival time measured by Kaplan-Meier plot

3. Presence and burden of luciferase-tagged cells in ipsilateral and contralateral lymph nodes as well as lungs.

Endpoints.

At the end of the experiments, mice were euthanized by ketamine/zylazine overdose followed by cervical dislocation. Triggers of euthanasia during the course of experiment were:

1. Weight loss >20%, a value determined based on the animals' weight prior to study initiation minus the weight of the tumor-bearing animal less the weight of the tumor;

2. Respiratory distress;

3. Skin ulceration;

4. One or more of the following signs of severe or chronic pain or distress: (i) lethargy, (ii) loss of inquisitiveness, (iii) dehydration, (iv) soiled hair coat, (v) closed eye lids, (vi) muscle wasting, (vii) hunched posture, (viii) ataxia (ix) soiled anogenital area, (x) circling.

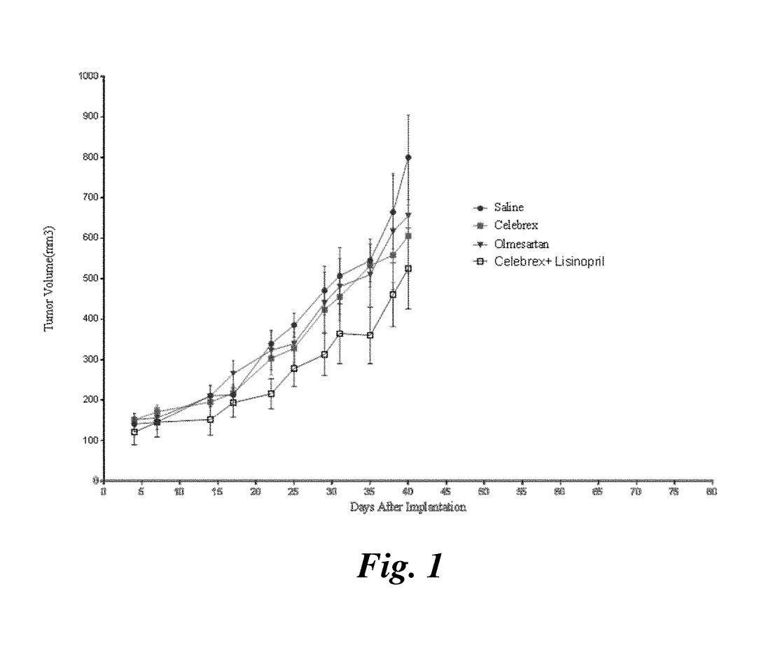

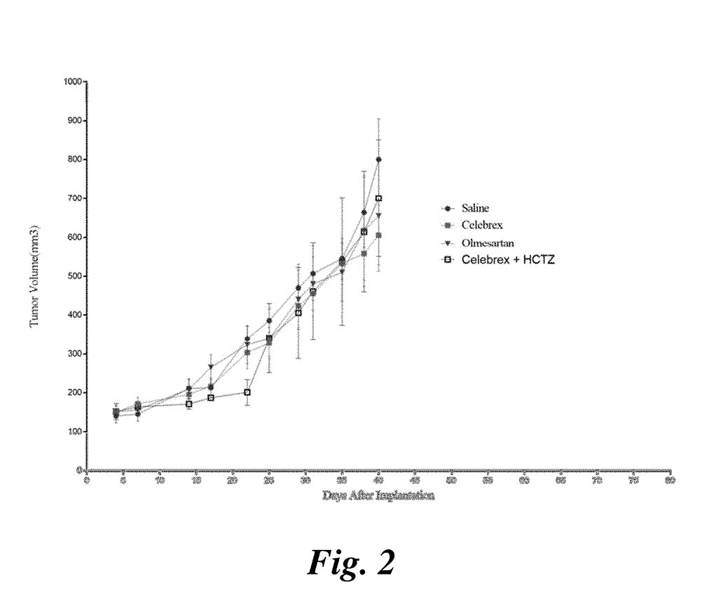

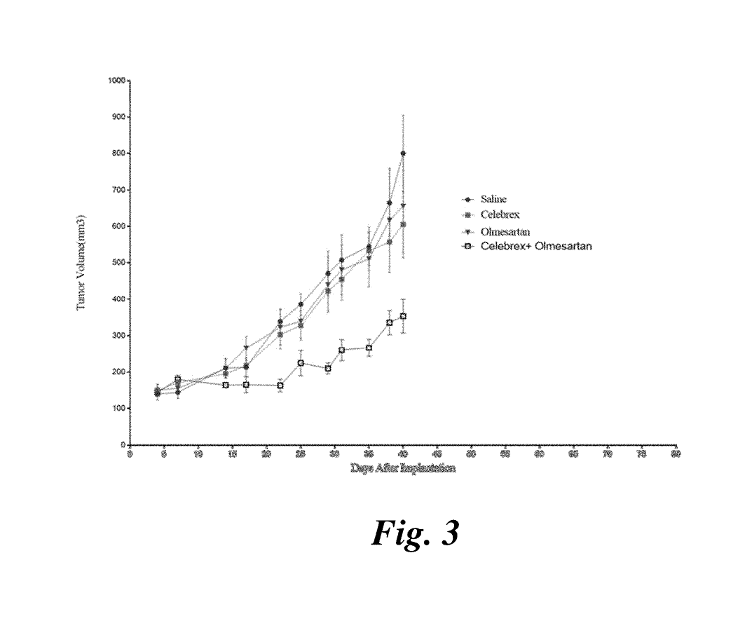

In the study, tumor growth was compared for celecoxib alone, three anti-hypertensive agents alone [lisinopril (an angiotensin converting enzyme inhibitor), hydrochlorothiazide (a diuretic), and olmesartan (angiotensin II receptor blocker)], and combinations of celecoxib with each of the antihypertensive agents. FIGS. 1-3 compare tumor volume as a function of time and administration of control (saline), celecoxib (Celebrex), olmesartan, and combinations of (1) celecoxib and lisinopril (Celebrex+Lisinopril) (FIG. 1), (2) celecoxib and hydrochlorothiazide (Celebrex+HCTZ) (FIG. 2), and (3) celecoxib and olmesartan (Celebrex+Olmesartan) (FIG. 3).

Only the combination of celecoxib and olmesartan showed tumor growth inhibition. See FIG. 3.

The results demonstrate that the combination of celecoxib and olmesartan is effective in inhibiting growth of tumors (MDA-MB-435 xenografts) and therefore the combination of celecoxib and olmesartan is effective in the treatment of breast and skin cancers. The results demonstrate that the combination of celecoxib and olmesartan is a synergistic combination for inhibiting tumor growth.

### Example 2

Background: Breast cancer and melanoma are associated with a high rate of lymphatic and hematogenous metastasis which results in high rate of cancer mortality. Inflammation and hypertension have recently emerged as causal factors for tumor progression and anti-hypertensive agents have been shown to reduce inflammation and suppress tumor growth and metastasis. Cyclooxygenase-2 is upregulated in most human tumors and is a potent inducer of cancer-associated inflammation that promotes tumor angiogenesis and lymphangiogenesis. This study evaluated a novel combination of a selective COX-2 inhibitor with three antihypertensive drugs to suppress tumor growth and metastasis in xenograft models.

Methods: Three anti-hypertensive drugs were evaluated in this study: i) Lisinopril [LIS], an inhibitor of angiotensin-1 converting enzyme (ACE); ii) Olmesartan medoxomil (OLM), an angiotensin II receptor blocker (ARB); and iii) Hydrochlorothiazide (HCTZ), a thiazide diuretic along with Celecoxib [CEL], a selective COX-2 inhibitor. CEL, LIS, OLM, and HCTZ were evaluated either alone or in combination for tumor growth suppression and metastatic spread in an orthotopic xenograft model of triple-negative inflammatory breast cancer/SUM149 and subcutaneous xenograft models of human melanoma/MDA-MB-435, glioblastoma/U87-MG, and triple-negative SUM159. Luciferase-tagged SUM149 and MDA-MB-435 cell lines were used to determine the incidence and the burden of locoregional and systemic spread. Mice were monitored twice a week for 9-16 weeks for percent weight loss, tumor volume and survival outcome. Metastatic tumor burden and incidence was measured as luciferase expression in lymph nodes and lungs and normalized to total protein.

Subcutaneous melanoma xenograft tumor model: MDA-MB-435-Luc: Female SCID mice were implanted with 4×106 MDA-MB-435-Luciferase tumor cells in 50% Matrigel subcutaneously on the left flank. Seven days post implantation, tumor-bearing mice in subgroups received either saline, Celecoxib (CEL, 25 mg/kg), Lisinopril (LIS, 15 mg/kg), Olmesartan (OLM, 30 mg/kg); or Hydrochlorothiazide (HCTZ, 10 mg/kg) as single agents or in combination with CEL for 5 consecutive days for 3 weeks.

Orthotopic human inflammatory breast cancer model: SUM149-RR: Female CB-17 SCID mice were implanted in the mammary fat pad with 4×106 SUM149-RR tumor cells in 50% Matrigel. Seven days post implantation, mice in subgroups of 5 received either saline, CEL (25 mg/kg), LIS (15 mg/kg), OLM (30 mg/kg) or HCTZ (10 mg/kg) as single agents or in combination with CEL for 5 days/wk for 3 weeks.

Subcutaneous human U87-MG glioblastoma model: 3×106 U87 glioblastoma cells were injected into the right flank of Balb/c nude mice and tumor-bearing (50-250 mm3) mice were randomized into 10 groups (10 mice/gp) and the antitumor activities of CEL (25 or 50 mg/kg, QDx21) and OLM (30 or 60 mg/kg, QDx21) or CEL plus OLM were evaluated following daily PO dosing. T/C (%) was calculated as the mean RTV (relative tumor volume) of treated tumors (T) divided by the mean RTV of control tumors (C)×100%. T/C (%)≦40% and P value <0.05 was considered to exhibit significant antitumor activity.

Tumors were monitored 2-3 times per week with digital calipers. Once tumors reached 1500 mm3, the mice were sacrificed. Tumors from MDA-MB-435-Luc and SUM149-RR were snap frozen and ipsilateral lymph nodes and lungs were collected for assessing metastatic burden and incidence by measuring luciferase expression per organ and normalized to total protein. Difference in incidence or burden as compared to the control was assessed by a Fisher exact test, or a Mann-Whitney test.

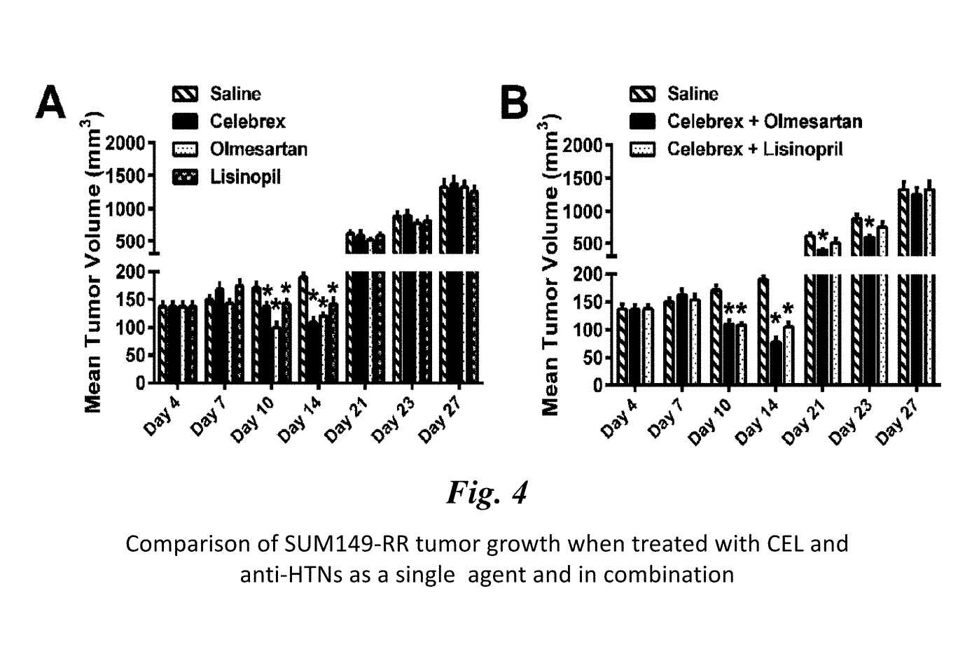

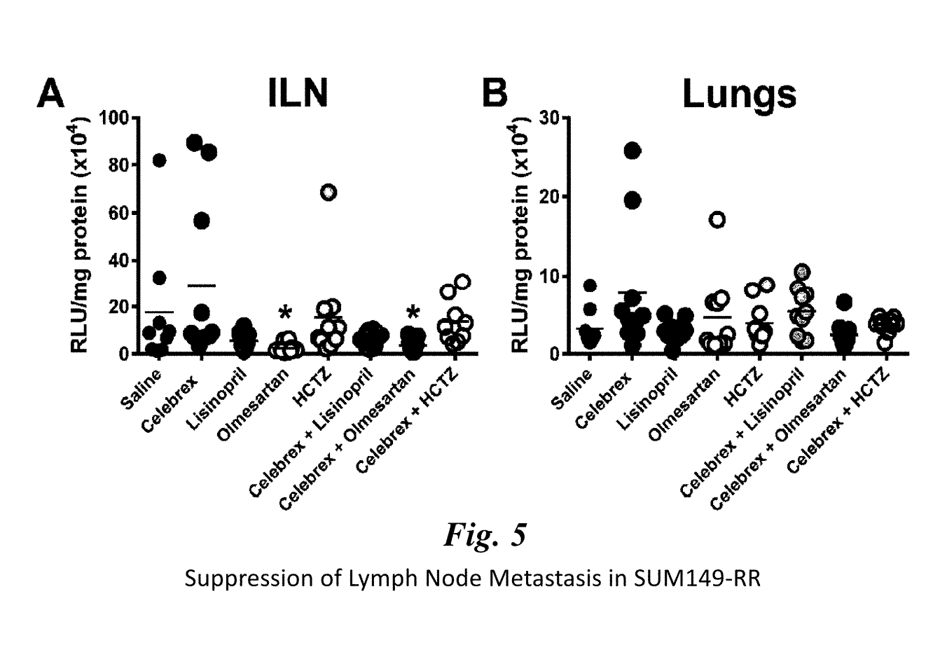

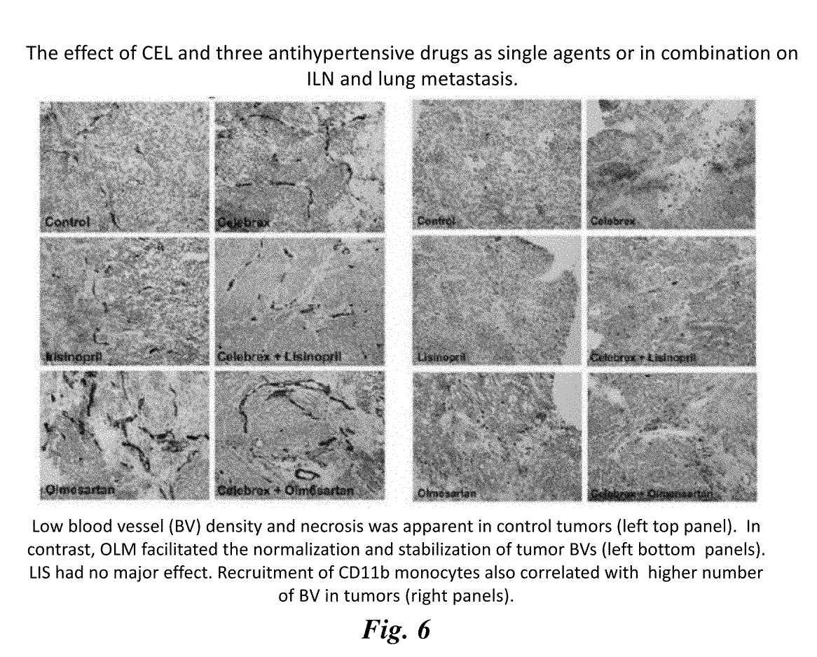

Results: In the SUM149 model, the saline treated control had an average tumor burden of 17.6±8.6×104 RLU per mg of protein in the ipsilateral lymph nodes (ILN). Two groups, OLM alone and CEL+OLM, had a statistically significant decrease in ILN burden. Olmesartan alone had a 7.1-fold decrease in tumor burden with an average of 2.4±0.6×104 RLU per mg of protein (p-value=0.01 by Mann-Whitney test). Similar trend was observed for LIS, but not for HCTZ. In the subcutaneous model, synergistic antitumor activity was observed with OLM (p=0.026) at low dose but not with LIS and CEL (p=ns). At high dose, LIS, OLM, and CEL showed significant inhibition of tumor growth but no synergy. When these agents were combined with Paclitaxel in the SUM159 model, there was only additive effect. HCTZ, an antihypertensive diuretic which has no direct impact on the vascular wall had no effect on tumor growth (see FIGS. 4-6).

These preclinical data strongly suggest a hitherto unappreciated role of ACE/ARB in tumor growth control.

### Example 3

Methods: Three anti-hypertensive drugs (Lisinopril [LIS], Olmesartan medoxomil [OLM] and Hydrochlorothiazide [HCTZ]) were evaluated for tumor growth suppression and metastatic spread in xenograft models of inflammatory breast cancer/SUM149, melanoma/MDA-MB-435 and glioblastoma/U87. Luciferase-tagged SUM149 and MDA-MB-435 cell lines were used to determine the incidence and the burden of locoregional and systemic spread. CB-17 SCID mice were implanted either subcutaneously (MDA-MB-435 and U87) or orthotopically (SUM149) with 4×10⁶ cancer cells and oral administration of test agents for 3 weeks was initiated on post-tumor inoculation Day 7. Mice were monitored twice a week for 9-16 weeks for percent weight loss, tumor volume and survival outcome. Metastatic tumor burden and incidence was measured as luciferase expression in lymph nodes and lungs and normalized to total protein.

Subcutaneous human melanoma xenograft tumor model: MDA-MB-435-Luciferase: Female SCID mice were implanted with 4×106 MDAMB-435-Luc tumor cells in 50% Matrigel subcutaneously on the left flank. Seven days post implantation, tumor-bearing mice in subgroups received either saline, Lisinopril (LIS, 15 mg/kg), Olmesartan (OLM, 30 mg/kg); or Hydrochlorothiazide (HCTZ, 10 mg/kg) for 5 consecutive days for 3 weeks.

### Orthotopic Human Inflammatory Breast Cancer Model SUM149-RR:

Female CB-17 SCID mice were implanted in the mammary fat pad with 4×106 SUM149-RR tumor cells in 50% Matrigel. Seven days post implantation, mice in subgroups of 5 received either saline, LIS (15 mg/kg), OLM (30 mg/kg) or HCTZ (10 mg/kg) for 5 days/wk for 3 weeks.

Subcutaneous human U87-MG glioblastoma model: 3×106 U87 glioblastoma cells were injected into the right flank of Balb/c nude mice and tumor-bearing (50-250 mm3) mice were randomized into 10 groups (10 mice/group) and the antitumor activities of OLM were evaluated following daily PO administration (30 or 60 mg/kg, QDx21). T/C (%) was calculated as the mean RTV (relative tumor volume) of treated tumors (T) divided by the mean RTV of control tumors (C)×100%. T/C (%)≦40% and P value <0.05 was considered to exhibit significant antitumor activity.

Tumors were monitored 2-3 times per week with digital calipers. Once tumors reached 1500 mm3, the mice were sacrificed. The tumor was snap frozen and ipsilateral lymph nodes and lungs were collected for assessing metastatic burden and incidence by measuring luciferase expression per organ and normalized to total protein. Difference in incidence or burden as compared to the control was assessed by a Fisher exact test, or a Mann-Whitney test.

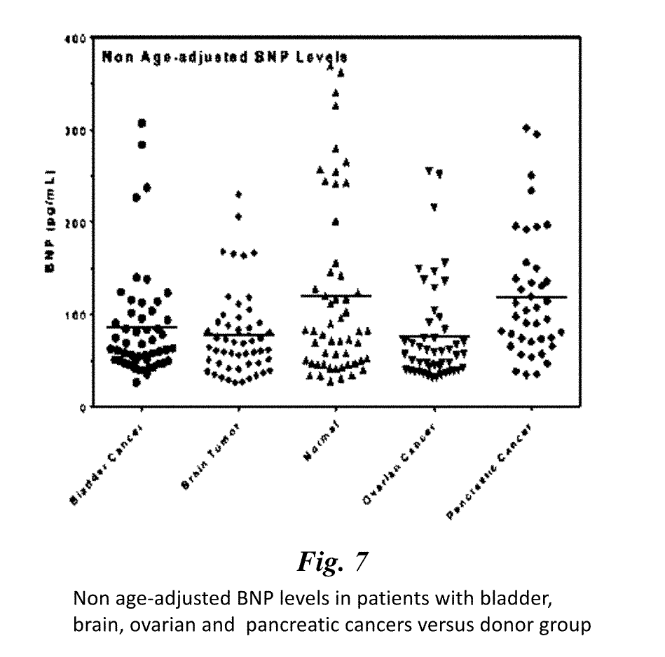

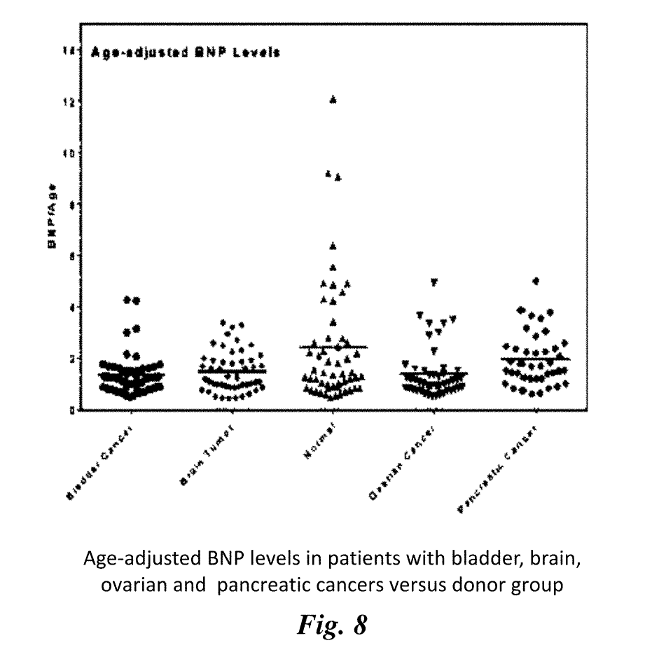

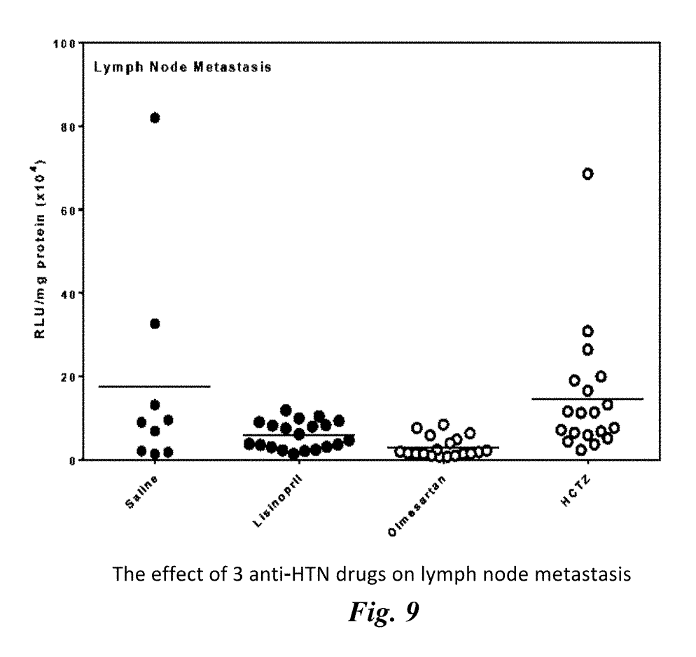

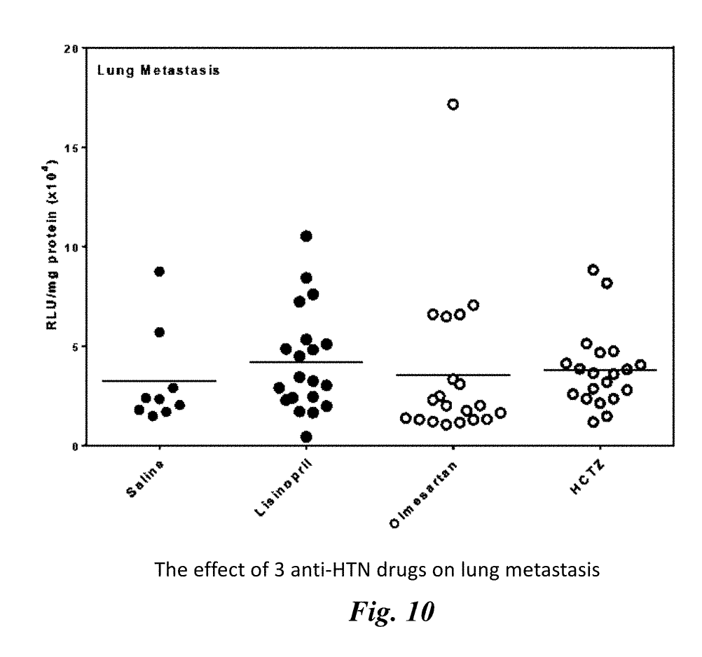

Results: Both LIS and OLM showed significant inhibition of tumor growth. HCTZ had no effect on tumor growth. OLM consistently resulted in significant (50-61%) inhibition of tumor growth in all three xenograft models in the SUM149 model, OLM induced a 5 to 7-fold reduction in metastatic tumor burden and reduced lymph node burden by 70 to 80% (17.6±8.6×10⁴ vs. 2.4±0.6×10⁴ RLU/mg of protein, P=0.01). Similar to OLM, LIS also resulted in significant reduction in tumor burden in MDA-MB-435 model. OLM and LIS lower the blood pressure by directly decreasing the vascular resistance unlike HCTZ, an antihypertensive diuretic which has no direct impact on the vascular wall suggesting the presence of a relationship between increased blood vascular resistance and cancer (see FIGS. 7-10).

Our finding with BNP suggested that a high vascular resistance tumor environment is conducive to tumor growth. Consistent with that hypothesis, the treatment with ACE/ARB, that directly act on the vasculature to reduce vascular resistance, inhibited tumor growth and metastasis. These data strongly suggest a hitherto unappreciated role of vascular resistance on tumor growth.

It is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A method of treating cancer, comprising administering a therapeutically effective amount of olmesartan or a pharmaceutically acceptable salt thereof, and celecoxib or a pharmaceutically acceptable salt thereof, to a subject in need thereof.

2. A method of treating breast cancer, skin cancer, or glioblastoma, comprising administering a therapeutically effective amount of olmesartan or a pharmaceutically acceptable salt thereof, and celecoxib or a pharmaceutically acceptable salt thereof, to a subject in need thereof.

3. (canceled)

4. (canceled)

5. The method of claim 2, wherein the olmesartan and celecoxib are administered concurrently as a pharmaceutical composition.

6. The method of claim 2, wherein administering the therapeutically effective amount of olmesartan and celecoxib comprises administering a fixed dose combination comprising olmesartan and celecoxib.

7. The method of claim 2, wherein olmesartan and celecoxib are administered orally.

8. The method of claim 2, wherein the subject is human.

9. A method of treating a cancer characterized by abnormal activation of COX-2, comprising administering a therapeutically effective amount of olmesartan or a pharmaceutically acceptable salt thereof, and celecoxib or a pharmaceutically acceptable salt thereof, to a subject in need thereof.

10. The method of claim 9, wherein the olmesartan and celecoxib are administered concurrently as a pharmaceutical composition.

11. The method of claim 9, wherein administering the therapeutically effective amount of olmesartan and celecoxib comprises administering a fixed dose combination comprising olmesartan and celecoxib.

12. The method of claim 9, wherein olmesartan and celecoxib are administered orally.

13. The method of claim 9, wherein olmesartan and celecoxib are administered intravenously.

14. The method of claim 9, wherein the cancer is selected from the group consisting of breast cancer, skin cancer, and glioblastoma.

15. (canceled)

16. (canceled)

17. The method of claim 9, wherein the subject is human.

18. The method of claim 9 further comprising assay of COX-2 to detect abnormal activation of COX-2 in a sample obtained from the subject containing cancer cells.

19. The method of claim 9 further comprising assay of angiotensin II receptor to detect abnormal level of angiotensin II receptor in a sample obtained from the subject containing cancer cells.

20. A pharmaceutical composition, comprising olmesartan or a pharmaceutically acceptable salt thereof, and celecoxib or a pharmaceutically acceptable salt thereof.

21. (canceled)

22. The pharmaceutical composition of claim 20, wherein the pharmaceutically acceptable carrier is a solid and the composition is a fixed dose combination.

23. A method of modulating vascular resistance in a cancer subject in need thereof, the method comprising the step of administering to the subject a therapeutically effective amount of an angiotensin receptor blocker (ARB) or an angiotensin converting enzyme (ACE) inhibitor, thereby reducing tumor growth in the subject in need thereof.

24. The method of claim 23, wherein the ACE inhibitor is selected from the group consisting of benazepril, captopril, enalapril, fosinopril, moexipril, perindopril, quinapril, ramipril, trandolapril, and lisinopril.

25. (canceled)

26. The method of claim 23, wherein the ARB is selected from the group consisting of eprosartan, azilsartan medoxomil, valsartan, telmisartan, losartan, candesartan, irbesartan, and olmesartan.

27. (canceled)

28. The method of claim 23, wherein the cancer is selected from the group consisting of melanoma, glioblastoma, ovarian cancer, bladder cancer, and breast cancer.

29. (canceled)

30. (canceled)

31. (canceled)

32. (canceled)

33. (canceled)

34. The method of claim 23, wherein the ACE inhibitor or ARB is administered concurrently with chemotherapeutic, hormonal, surgical, or radiation treatment.

35. The method of claim 23, wherein the cancer subject has not been treated with a therapeutically effective amount of an angiotensin receptor blocker (ARB) or an angiotensin converting enzyme (ACE) inhibitor.
